(19) United States
(12) Patent Application Publication
(10) Pub. No.: US 2025/0241766 A1
(43) Pub. Date: Jul. 31, 2025

(54) Spinal Implant With Porous And Solid Surfaces

(71) Applicant: Stryker European Operations Holdings LLC, Portage, MI (US)

(72) Inventors: Steven Willis, Jackson, NJ (US); Justyna Zielinska, Bridgewater, NJ (US); Robin Stamp, Montclair, NJ (US); Chau Ngo, Secaucus, NJ (US)

(21) Appl. No.: 19/072,404

(22) Filed: Mar. 6, 2025

Related U.S. Application Data
(62) Division of application No. 17/225,393, filed on Apr. 8, 2021, now Pat. No. 12,268,613.

(51) Int. Cl. A61F 2/44; A61F 2/30; A61F 2/46; B22F 10/20

(52) U.S. Cl. CPC A61F 2/447; A61F 2/3094; A61F 2/44; A61F 2/4455; B22F 10/20; A61F 2002/30011; A61F 2002/30593; A61F 2002/30904; A61F 2002/3092; A61F 2002/3093; A61F 2002/30985; A61F 2/4611

(57) ABSTRACT

A spinal implant including porous and solid portions is disclosed. The implant includes porous portions on upper and lower surfaces and in an interior thereof. Methods of manufacturing and implanting such implants are also disclosed.

### CROSS-REFERENCE TO RELATED APPLICATION

The present application is a divisional of U.S. application Ser. No. 17/225,393, filed Apr. 8, 2021, which is a continuation of U.S. application Ser. No. 16/219,254, filed on Dec. 13, 2018, now U.S. Pat. No. 11,000,386, which is a continuation of U.S. application Ser. No. 14/994,749, filed on Jan. 13, 2016, now U.S. Pat. No. 10,182,923, and claims the benefit of U.S. Provisional Patent Application No. 62/103,276, filed on Jan. 14, 2015, the disclosures of which are hereby incorporated herein by reference in their entirety.

### BACKGROUND OF THE INVENTION

The present invention relates to spinal surgery, namely, implants utilized in fusing adjacent intervertebral bodies or the replacement of a vertebral body.

Back pain can be caused by many different maladies, not the least of which are problems that directly impact the intervertebral discs of the spine. Typical disc issues include, inter alia, degeneration, bulging, herniation, thinning and abnormal movement. One method of treatment of such disc problems that has been widely utilized in the field of spinal surgery is a spinal fusion procedure, whereby an affected disc is removed, and the adjacent vertebral bodies are fused together through the use of interbody spacers, implants or the like. In some instances, it may also be necessary to remove and replace an entire vertebral body. This is often accomplished through the use of a larger implant that acts to fuse together the vertebral bodies adjacent the removed vertebral body.

The aforementioned implants often rely upon mechanical features to ensure engagement between the devices and the bone of the existing vertebral bodies. This coupled with the normal compressive load of the spine acts to keep the implant in place until bone can grow from the existing vertebral bodies into and through the implant. To encourage the bone growth, the implants are often pre-loaded with bone growth promoting material and thereafter placed into the spine. Bone growth promoting material may include naturally occurring bone, artificial materials or the like.

To further ensure a strong implant-bone connection, some existing implants include an area formed of porous material that allows bone to grow into it. Although there is little doubt that the bone growth into the implant is beneficial in maintaining an implant in place, these implants are often very difficult (and thusly, expensive) to manufacture. Additionally, existing implants that implement porous material do so in a limited manner. Often times, because of manufacturing or strength concerns or the like, the porous material is limited to a thin layer covering the upper and lower surfaces of the implant, which only allows for a small amount of bone to grow into the implant.

Therefore, there exists a need for an improved spinal implant that employs a significant amount of porous material, yet remains cost efficient and maintains the necessary strength required of a spinal implant.

### BRIEF SUMMARY OF THE INVENTION

A first aspect of the present invention is a spinal implant including an upper surface including a first porous portion and first solid portion, a lower surface including a second porous portion and a second solid portion and a cavity formed through the upper and lower surfaces, the cavity including a third porous portion.

Other embodiments according to the first aspect may include a nose having a solid exterior, a hollow area and a porous region. At least one serration may be included on each of the upper and lower surfaces. The serration(s) may include a solid tip, a solid root and a porous section. The implant may further include first and second side walls extending between the upper and lower surfaces, the side walls including a solid exterior surface and a porous interior surface. The first and second side walls may each include lateral windows. The lateral windows may reduce the stiffness of the implant and may be tapered. The implant may also include a threaded opening at a rear end. Implants according to the present invention may be constructed of any material suitable for implantation in the body of a patient, for instance, a metal such as titanium. The implants can be configured for insertion from various aspects, e.g., a posterior approach, a lateral approach or an anterior approach. The implant may include a nose that facilitates the insertion of the implant in a first orientation and rotation to a second orientation. The implant may be constructed from an additive manufacturing process, and may be machined to create smooth surfaces.

### BRIEF DESCRIPTION OF THE DRAWINGS

A more complete appreciation of the subject matter of the present invention and of the various advantages thereof can be realized by reference to the following detailed description in which reference is made to the accompanying drawings in which:

FIG. 1 is a front perspective view of an implant according to one embodiment of the present invention.

FIGS. 2A and 2B are rear perspective views of the implant of FIG. 1.

FIG. 3 is a side view of the implant of FIG. 1.

FIGS. 4A and 4B are top views of the implant of FIG. 1.

FIG. 5 is a rear view of the implant of FIG. 1.

FIG. 6 is a cross-sectional view of the implant of FIG. 1 take along line 6-6 of FIG. 5.

FIG. 7 is an enlarged cross-sectional view of serrations of the implant of FIG. 1.

FIGS. 8A-8B are cross-sectional views of the implant of FIG. 1 take along lines 8A-8A and 8B-8B of FIGS. 4A and 4B, respectively.

FIGS. 9A-9C are views illustrating a constructed version of the implant of FIG. 1.

FIG. 10 is a fluoroscopic view of an implanted implant of FIG. 1.

FIGS. 11A-11C are views of implants according to other embodiments of the present invention.

FIGS. 12A-12C are views of implants according to other embodiments of the present invention.

FIGS. 13A-13B are views of implants according to other embodiments of the present invention.

FIGS. 14A-14B are views of implants according to other embodiments of the present invention.

FIGS. 15A-15C are views of implants according to other embodiments of the present invention.

FIG. 16 depicts yet another implant according to another embodiment of the present invention.

### DETAILED DESCRIPTION

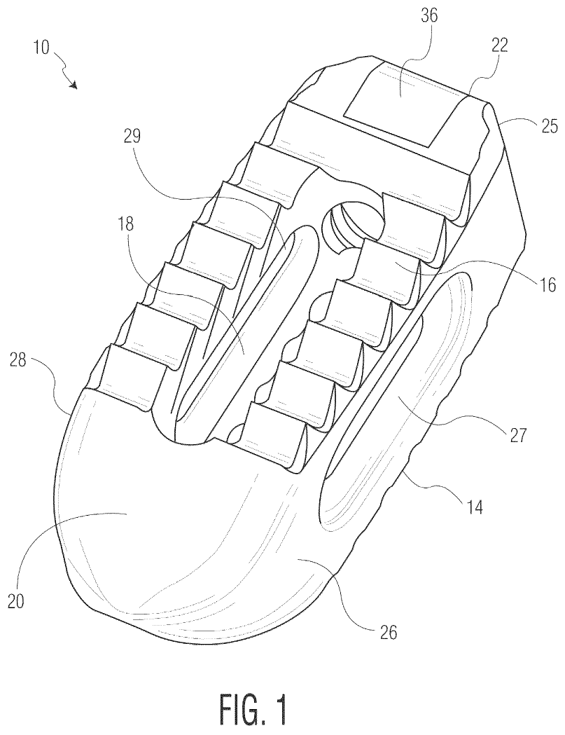

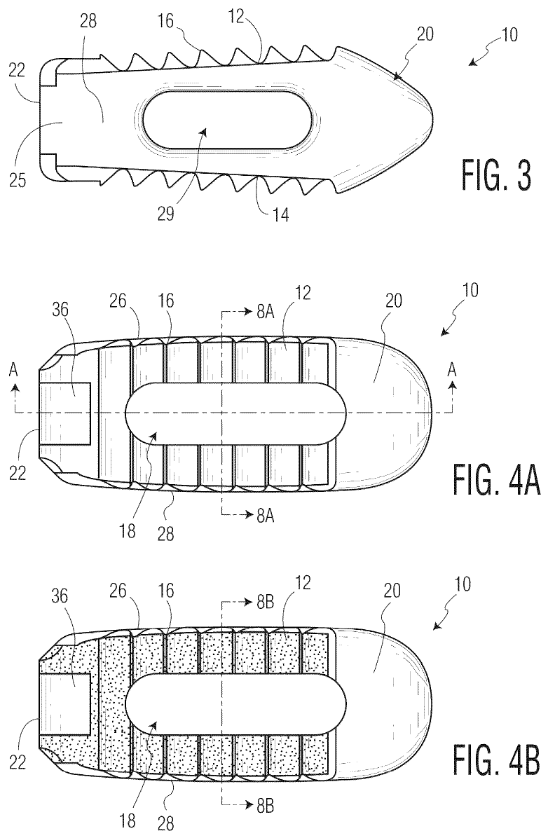

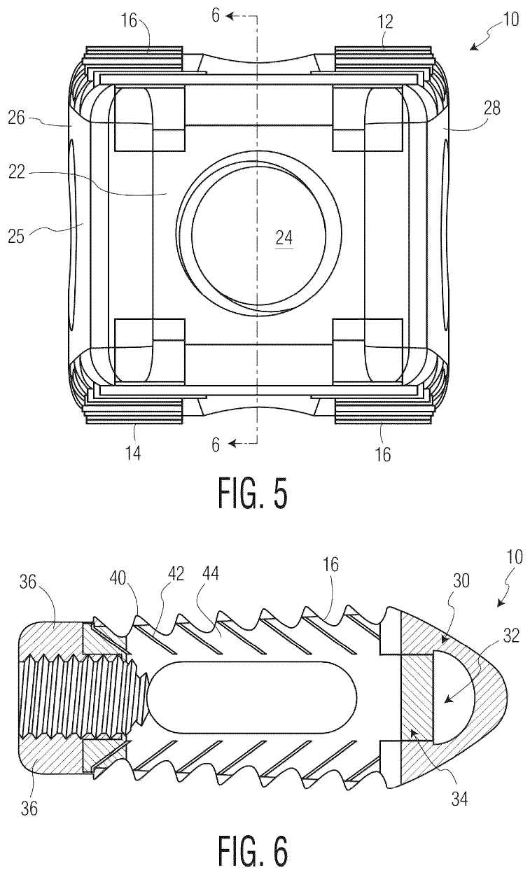

An implant 10 according to a first embodiment of the present invention is depicted in FIGS. 1-10. Implant 10 is shown as an implant suitable for implantation from a posterior approach. However, as will be readily apparent from the below discussion pertaining to other embodiments, the present invention is not limited to any particular type of implant design. Rather, it is contemplated that certain features of the present invention can be implemented in different types of implants. For instance, implants according to the present invention can be adapted for implantation from anterior or lateral aspects of the patient, as will be discussed below. Moreover, although disclosed as being constructed of metallic materials, it is contemplated that implants according to the present invention may be constructed of polymeric materials such as PEEK or the like. Additionally, each of the embodiments shown in the drawings are designed for placement between adjacent vertebral bodies. However, it is contemplated that implants in accordance with the present invention may be designed for use as vertebral body replacements.

Implant 10 is shown including upper and lower surfaces 12 and 14, respectively. Each surface includes a plurality of serrations 16 at least covering a portion of the surface. While a specific serration design is depicted in the drawings and described in more detail below, many different serration designs can be employed. Implant 10 also includes a cavity 18 formed through a central portion of the implant and each of surfaces 12 and 14. Cavity 18 can be sized and shaped differently from what is shown and can be located in other locations of implant 10. Cavity 18 is preferably designed so that bone growth promoting materials can be contained therein to promote bone growth through the implant.

Implant 10 also includes a wedge nose 20, a rear end 22 with a threaded opening 24 and a chamfer interface 25, and sidewalls 26 and 28 through which lateral windows 27 and 29, respectively are formed. Wedge nose 20 is sized and shaped so as to distract vertebral bodies during insertion of the implant into the intervertebral space. Threaded opening 24 and chamfer interface 25 are configured to cooperate with an insertion tool (not shown in detail). Lateral windows 27 and 29 act to both reduce the stiffness of implant 10 and allow for visualization through the lateral aspect of the implant under fluoroscopy imaging. Of course, the specific sizes and shapes of these elements may vary in other embodiment implants in accordance with the present invention, including certain embodiments discussed below. For instance, certain of the surfaces of implant 10 are shown as smooth and rounded to reduce the potential for soft tissue damage during an implantation procedure, but can be configured differently.

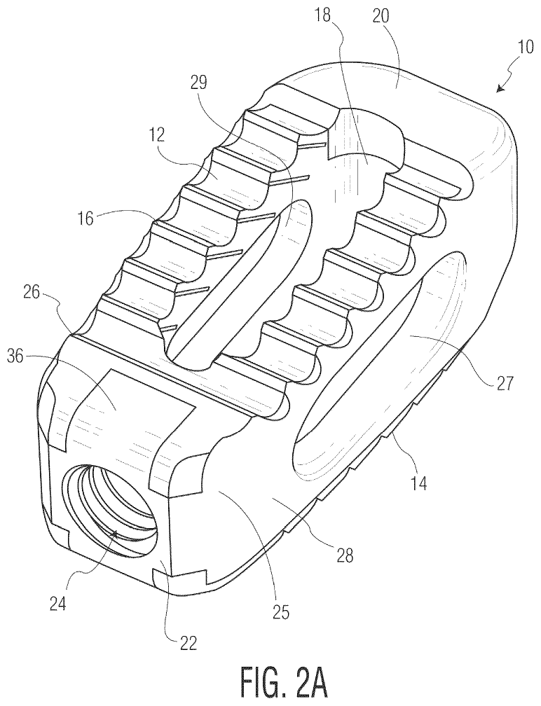

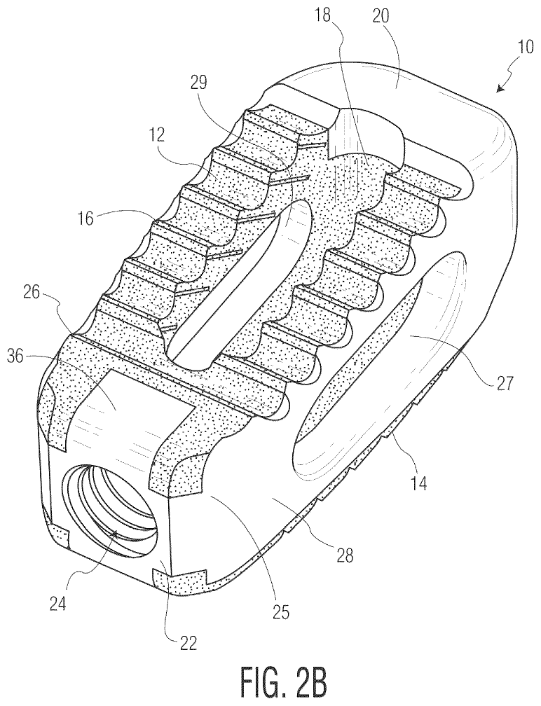

Implant 10 is formed of both solid and porous portions. The porous portions are located on upper and lower surfaces 12, 14, as well as on certain of the internal surfaces of the implant, which allows for bone to grow into a significant portion of the implant. This can best be seen in FIGS. 2B, 4B, 8B, and 9A-9C, where the porous surfaces of implant 10 are shown with different shading. In one embodiment, the porous surfaces have an average pore diameter between 100-1000 microns with a 30-80% porosity, while a preferred embodiment would have a porosity between 55-65%. The porous surfaces may also have any thickness, for instance between 500-4500 microns, and preferably between 500-1500 microns. This results in a surface that is both strong enough for use in a spinal implant and maximizes bone growth potential. The porous portions of implant 10, as well as the solid portions, can be created through the use of a 3D printing process such as is disclosed in U.S. Pat. Nos. 7,537,664 and 8,147,861; U.S. Patent Application Publications Nos. 2006/0147332, 2007/0142914, 2008/0004709; and U.S. patents application Ser. Nos. 13/441,154 and 13/618,218, the disclosures of which are hereby incorporated by reference herein. It is also contemplated to form any porous portion via another known or hereafter developed procedure, such as laser etching.

With specific reference to FIGS. 2B, 4B and 6-8B, the location of the porous and solid portions of implant 10 will be discussed. In the solid model views of FIGS. 2B and 4B, the porous portions of the implant are shown as darker sections, while the solid portions are depicted in lighter material. The cross-sectional views of FIGS. 6-8B on the other hand depict these portions with different cross hatching. For instance, nose 20 includes a solid, smooth exterior construction. The use of solid metal in this section allows for it to withstand impaction loads during an insertion process, as well as for visualization of its location under fluoroscopy or other imaging. It is shown in FIG. 6 that nose 20 in actuality includes a solid portion 30, a hollow area 32 and a porous region 34. Solid portion 30 is designed to provide the necessary support discussed above, while hollow area 32 is provided in order to decrease the radioopacity of the nose and improve visualization under fluoroscopy imaging. Porous region 34, as will be discussed more fully below, extends into the area within cavity 18. It is contemplated that in other embodiments, porous region 34 may extend partially or completely into hollow area 32. This still acts to decrease the radioopacity of the nose, which improves visualization, but also improve the cleanability, sterilization and powder removal from the implant during processing.

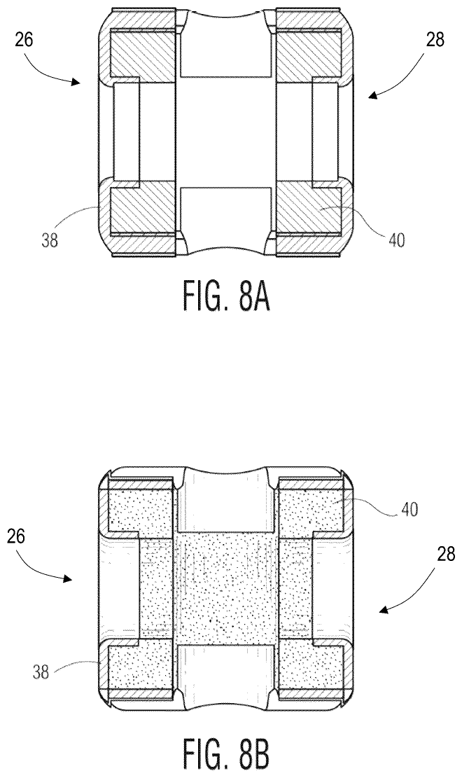

Like nose 20, a significant portion of rear end 22 is formed of solid material, so as to facilitate a strong connection with an insertion tool (not shown in detail). In particular, it is noted that while certain portions of the upper and lower surfaces 12, 14 at the rear end are porous, sections 36 are formed solid as they overlie threaded opening 24. This construction adds the necessary stability to the opening that is required for a solid connection with the insertion tool. Moreover, side walls 26, 28 are, as is best shown in FIGS. 8A and 8B, formed solid on an exterior of implant 10 and porous in an interior thereof. Specifically, with reference to FIG. 8B, the side walls include solid portions 38 and porous portions 40. Again, the inclusion of solid portions 38 provides stability to implant 10. However, as is mentioned above, lateral windows 27, 29 reduce the stiffness of the implant. Solid portions 38 may be any thickness, for instance, within the range of 0.25 mm to 0.5 mm. The solid portions also serve to provide a smooth exterior surface to the implant, which reduces tissue damage during implantation. It is noted that in certain embodiments, material may be machined off of any of the surfaces to create a smooth surface finish, which may further prevent tissue damage during implantation. This is especially true in connection with implanted formed by 3D printing processes, as such often result in even solid portions having a rougher surface finish.

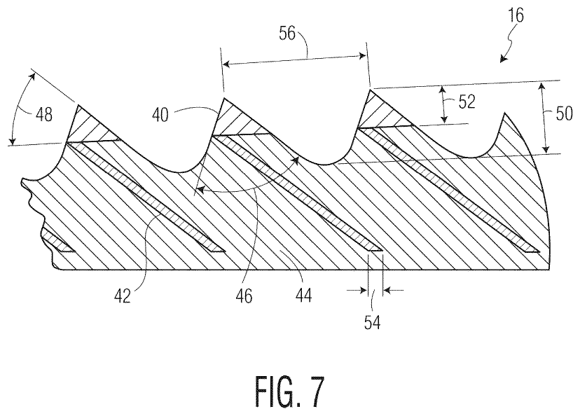

Aside from the above discussed portions that are formed solid, the majority of the remainder of implant 10 is formed porous. Most notably, upper and lower surfaces 12, 14 are largely porous, especially in the portions having serrations 16. However, the serrations themselves include some solid portions. With reference to FIGS. 6 and 7, serrations 16 include solid tips 40 and solid roots 42 forming solid (non-porous) struts, with the remainder of their construction including porous sections 44 forming porous struts. Solid tips 40 not only provide a strong leading surface for engagement with bone, but also prevent fracture of a porous surface from occurring upon such engagement. Specifically, since the individual components (e.g., struts) of the porous surfaces of implant 10 may not necessarily converge to a point, they may fracture upon application of a force like what would be transmitted to serrations 16 during implantation. Solid core 42 also acts to strengthen serrations 16, by essentially providing a strong foundation for porous sections 44. As shown in FIGS. 4A and 4B, at least one serration 16 including solid tips 40 and solid roots 42 extends across side walls 26 and 28 to form a solid (non-porous) cross-strut.

The particular shape of serrations 16 is also designed to create a strong initial implant-bone connection, while also allowing for easy insertion of implant 10 into the space between vertebrae. In order to resist back-out of implant 10, serrations 16 are oriented at an angle 46 (see FIG. 7). This angle may be any value, although a value within the range of 60 to 80 degrees is preferable. The angle 48 (see FIG. 7) of solid tips 40 is preferably in the range of 30 to 50 degrees. The height 50 of serrations 60 may be within the range of 0.5 mm to 1.5 mm, while the height 52 of solid tips 40 is dependent upon height 50, but preferably is within the range of 0.25 mm to 0.5 mm. Solid core 42 has a thickness 54, preferably 0.1 mm to 0.3 mm thick. The overall pitch 56 of serrations 16 is preferably between 1.25 mm and 2 mm.

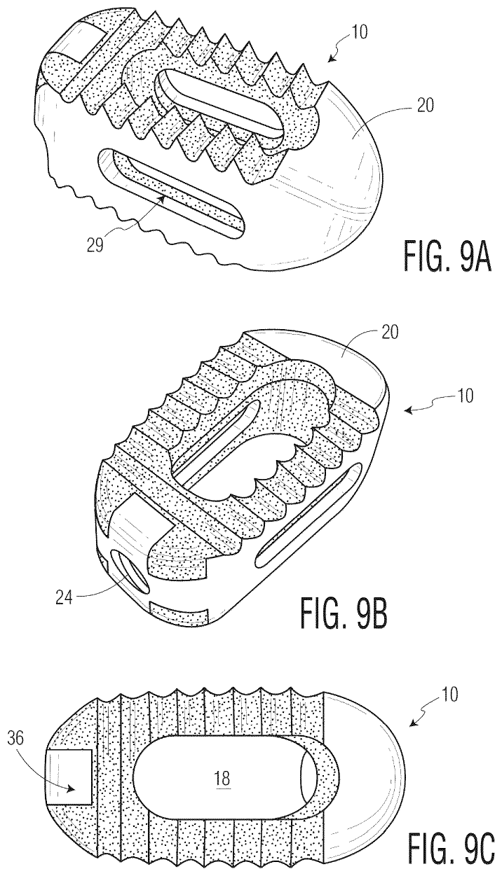

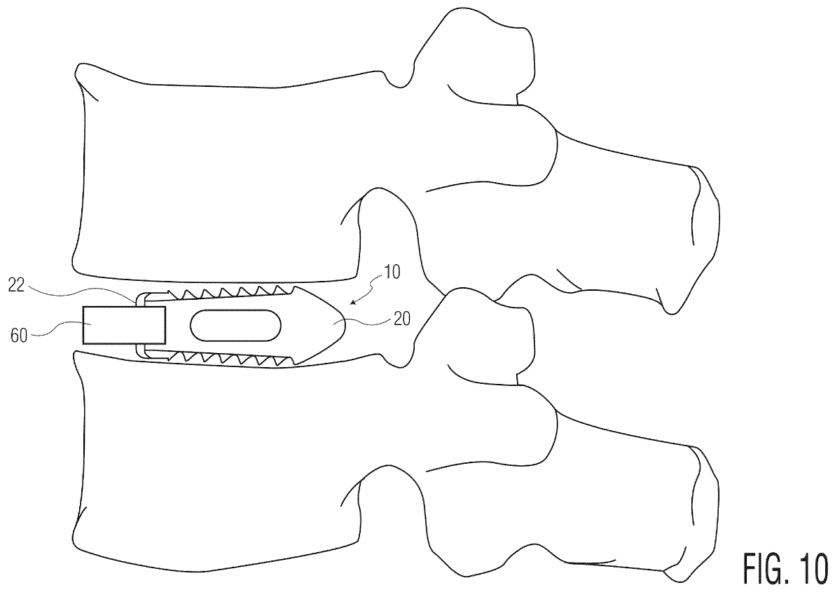

The interior of cavity 18 is largely constructed of porous material, which allows for bone growth in this section as well, and hence fusion through implant 10. This construction has the added benefit of also reducing stiffness of the implant, like lateral windows 27, 29. A fully constructed implant 10 is depicted in FIGS. 9A-9C. As shown, the various solid and porous portions of the implant appear differently to the naked eye. The particular prototype shown in those figures was created via a 3D printing process referred to as additive manufacturing, utilizing a titanium material. FIG. 10 is a fluoroscopic image of implant 10 while in position between two adjacent vertebral bodies. In the particular image shown there, implant 10 is engaged with an insertion tool 60, although the specifics of that tool cannot be seen.

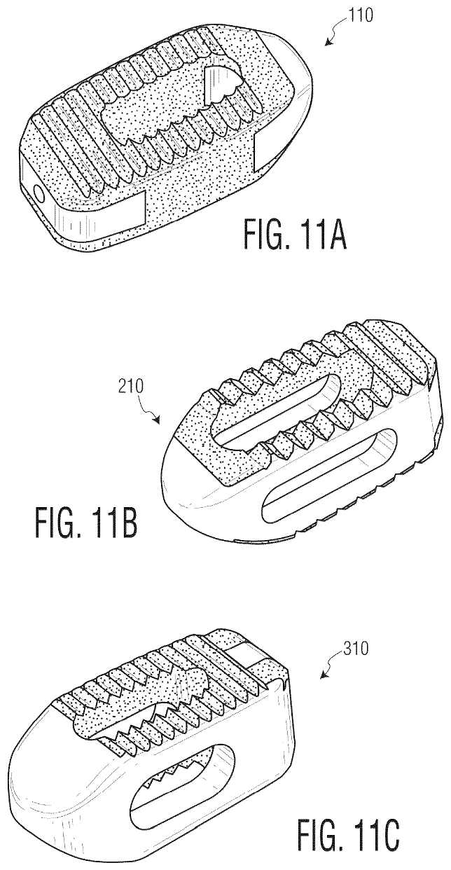

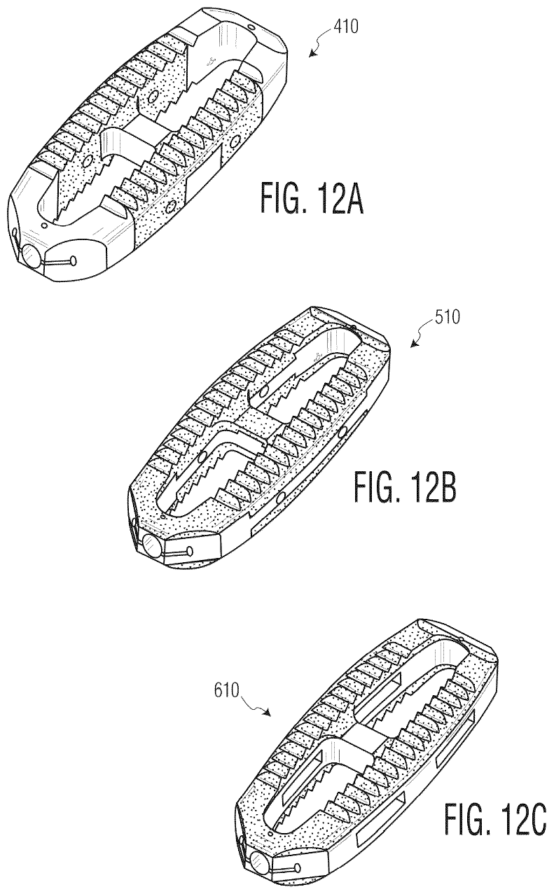

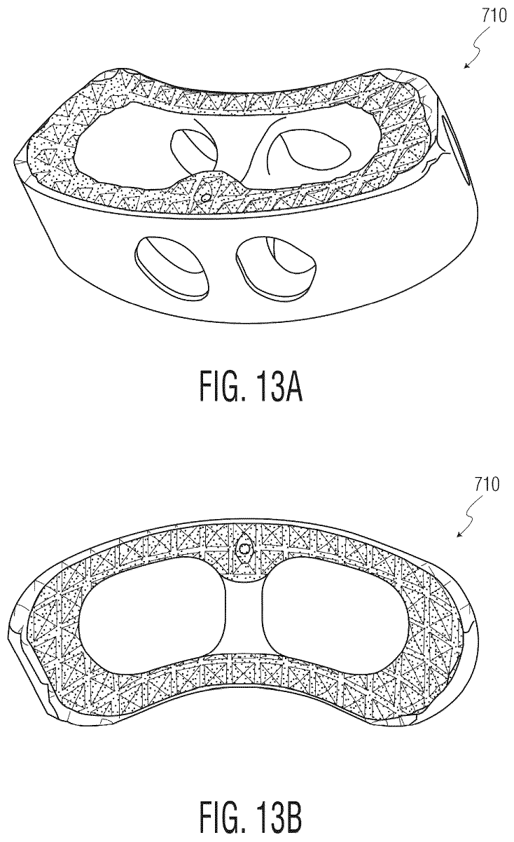

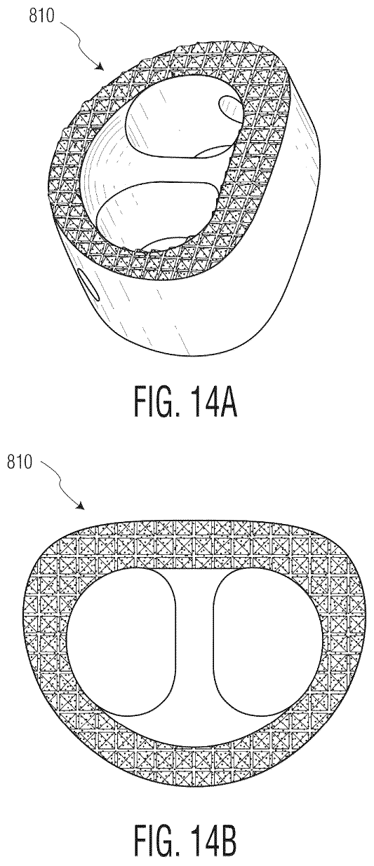

FIGS. 11A-11C depict different embodiment implants 110, 210 and 310, respectively that are each suitable for implantation from a posterior approach, like implant 10. FIGS. 12A-12C depict different embodiment implants 410, 510 and 610, respectively that are each suitable for implantation from a lateral approach. FIGS. 13A-13B depict an implant 710 suitable for implantation from a posterior lateral approach. FIGS. 14A-14B depict an implant 810 suitable for implantation from an anterior approach. Among other ways, those implants differ from implant 10 and each other in the manner in which their solid and porous portions are dispersed throughout the design. Again, solid portions are shown in lighter shading and porous portions are shown in darker shading. These various implant embodiments demonstrate that implants in accordance with the present invention may vary both in their size and shape, as well as in the configuration of their porous and solid portions.

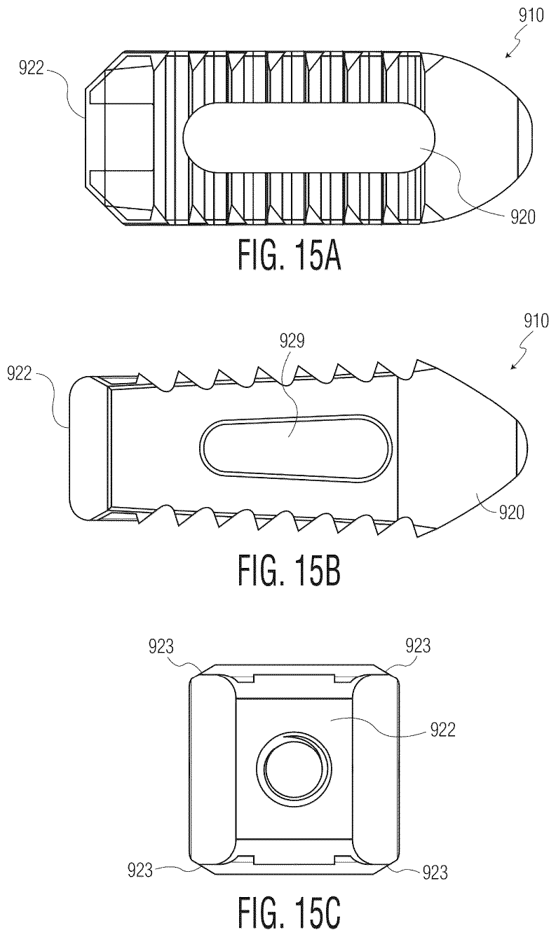

FIGS. 15A-15C depict an implant 910 similar to that of implant 10, albeit with certain specific differences. For instance, nose 920 includes sidewalls (best shown in FIG. 15A) that exhibit an increased angle from that of nose 20. This particular design allows for the implant to be inserted in an orientation that is rotated ninety degrees from the traditional insertion orientation of such an implant. Thereafter, implant 910 is rotated, which may result in an additional distraction from that of the initial insertion. Implant 910 may also be provided with a feature, such as a dimple or the like (not shown), that helps to identify the correct final orientation of the implant. For instance, a dimple may be provided at rear end 922 so that the surgeon may easily identify the final orientation of the implant. Of course, any visual identifier could also be employed.

Implant 910 also includes differently shaped/oriented lateral windows 927, 929 (only window 929 is shown in FIG. 15B) from that of above-discussed windows 27, 29. As shown, windows 927, 929 extend along less of implant 910 than do windows 27, 29 along implant 10. Moreover, the height of windows 927, 929 taper in the same direction as does the height of implant 910. For implants that are not lordotic, the windows may be a constant height. Finally, implant 910 exhibits chamfered edges 923 (best shown in FIG. 15C) that are on the four sides of the implant to eliminate sharp edges an make the implant more suitable for implantation without tissue damage.

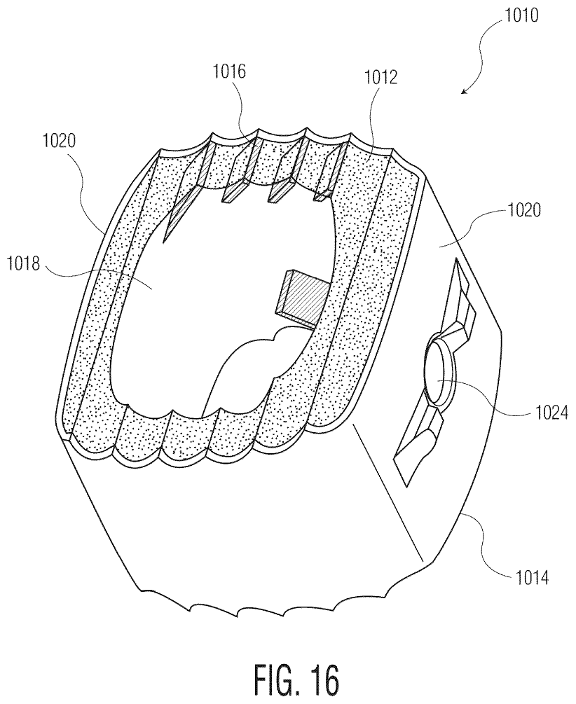

FIG. 16 depicts yet another embodiment according to the present invention, cervical implant 1010. This implant is particularly suited for implantation in a cervical area of the spine and includes many elements similar to those of the other embodiment implants. For instance, implant 1010 includes upper and lower surfaces 1012, 1014 which include serrations 1016 similar to those discussed above. Further, the cervical implant includes a tapered nose or leading end 1020 and a trailing end 1022 with an aperture 1024 for engaging an insertion tool. Although other embodiments may vary, implant 1010 is shown as having porous portions at the upper and lower surfaces 1012, 1014 that are similar to those discussed above.

In use, the various implants in accordance with the present invention may be implanted in a manner similar to existing spinal implants. For instance, an insertion tool (e.g., tool 60) may be coupled with the implant to guide the implant into place between vertebral bodies. Initial engagement of the implant with the vertebral bodies is achieved via mechanical coupling elements included on the implant (e.g., serrations 16). Thereafter, bone is permitted to grow into any porous sections on the implant. This bone growth may be promoted through the use of bone growth promoting substances, such as allograft materials placed within cavity 18. After some time, the porosity of the implant preferably allows for a stronger fusion than that of existing, nonporous implants.

In creating an implant such as implant 10, the aforementioned 3D printing process can be utilized (see e.g., FIGS. 9A-9C). Because of the construction of the implant, it may be beneficial to orient the construction in one manner or the like. For instance, it has been found that orienting the build so that nose 20 faces down (i.e., is built first) results in better serration 16 creation. Of course, the nose down orientation is only one of many that can be employed and the creation of implants according to the present invention is not to be so limited.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A spinal implant system comprising:
a spinal implant having an elongate body having:
a central longitudinal axis extending through the body;
a front section and a rear section, the rear section having a rear wall and an internally threaded opening;
an upper surface extending between the front section and the rear section;
a lower surface extending between the front section and the rear section;
a first side wall extending between the upper surface and the lower surface on a first side of the longitudinal axis; and
a second side wall extending between the upper surface and the lower surface on a second side of the longitudinal axis, the first side wall and the second side wall comprising a first recess and a second recess, respectively,
wherein an intersection of the first side wall and the upper surface comprises a first chamfered edge and wherein an intersection of the second side wall and the lower surface comprises a second chamfered edge,
wherein the rear wall is generally perpendicular to the first side wall and the second side wall; and
an insertion tool having first and second arms configured to contact the first and second chamfered edges,
wherein the insertion tool is removably attachable to the implant and adapted to insert the implant between adjacent vertebrae in a first orientation with the first and second side walls configured to contact the adjacent vertebrae and rotating the implant 90° to a second orientation with the superior and inferior surfaces configured to contact the adjacent vertebrae.

2. The spinal implant of claim 1, wherein the body includes porous and non-porous sections, one or more non-porous struts extending through the porous section from the first side wall to the second side wall.

3. The spinal implant of claim 2, wherein at least one of the non-porous struts extends beyond any of the upper or lower surfaces.
